(19) United States
(12) Patent Application Publication
(10) Pub. No.: US 2025/0027855 A1
(43) Pub. Date: Jan. 23, 2025

(54) BIOLOGICAL KIT FOR SEPARATING ELECTRONEGATIVE LOW DENSITY LIPOPROTEIN FROM SPECIMEN, REAGENT SOLUTION AND METHOD FOR SEPARATING ELECTRONEGATIVE LOW DENSITY LIPOPROTEIN FROM SPECIMEN

(71) Applicant: Kerth Corp., Milpitas, CA (US)

(72) Inventors: SHU-HSUAN LIU, Kaohsiung City (TW); Liang-Yin Ke, Kaohsiung City (TW); Jen-Yu Wei, Taipei City (TW)

(73) Assignee: Kerth Corp., Milpitas, CA (US)

(21) Appl. No.: 18/716,146

(22) PCT Filed: Jan. 3, 2022
(86) PCT No.: PCT/US2022/011057

§ 371 Date: Jun. 4, 2024

(51) Int. Cl. G01N 1/40

(52) U.S. Cl. CPC G01N 1/4044; G01N 1/4077; G01N 2001/4083

(57) ABSTRACT

A biological kit for separating electronegative low density lipoproteins is disclosed. The biological kit includes a first reagent, a second reagent, a third reagent and a fourth reagent. The first reagent includes water. The second reagent includes a first buffer, a second buffer and a salt. The third reagent includes the first buffer, the second buffer, an organic compound and the salt. The fourth reagent includes the first buffer and the second buffer.

### BACKGROUND OF THE INVENTION

### 1. Field of the Invention

The present invention generally relates to biological kit for separating electronegative low density lipoproteins (LDL-) from a specimen and a method for separating electronegative low density lipoproteins from a specimen. In particular, the present invention is directed to a use of a biological kit for separating electronegative low density lipoproteins from a specimen in the presence of reagents to greatly reduce the operation time.

### 2. Description of the Prior Art

Previous studies have shown that naturally occurring electronegative low-density lipoproteins may be an important risk factor for cardiovascular diseases. Accordingly, naturally occurring electronegative low-density lipoproteins need separating from the plasma for further study or for further investigation.

Because the electronegative low-density lipoproteins are a minor component in the plasma, it takes a lot of time, for the current approaches to separate the electronegative low-density lipoproteins from the plasma with undesirable quality. For example, the conventional method may take several steps to separate different components from plasma, and each step may take at least 24 hours to separate a specific lipoprotein. Specifically, with the conventional method, it may take at least 96 hours to sequentially separate all lipoprotein portions from a biological fluid. Furthermore, while rapid separation of plasma lipoprotein can be achieved through density gradient medium, such as iodixanol, the resulting lipoprotein portions are of undesirable quality and so it cannot be used to separate electronegative low-density lipoprotein. For example, due to iodixanol's properties, such as large molecular weight and the presence of organo-iodine, it cannot be efficiently removed through dialysis to yield a pure, iodixanol-poor lipoprotein portion. The inability to completely remove iodixanol from lipoprotein samples renders separation, further analysis, and quantification of electronegative low-density lipoproteins impossible. In light of these time-consuming separation steps and undesirable quality, a novel solution to provide better separation quality and a faster method to separate the electronegative low-density lipoproteins from the plasma are urgently needed to break the long operation time barrier to accelerate a scientific study or a medical investigation.

### SUMMARY OF THE INVENTION

Given the above, the present invention is capable of proposing a novel method to separate an iodixanol-poor and electronegative low density lipoprotein from a specimen to greatly reduce the operation time to overcome the problems in prior art. The present invention further proposes a novel method to separate a high density lipoprotein from a specimen for the further scientific study, the medical identification or screening of metabolism-related disorders. The present invention also proposes a convenient biological kit for separating an electronegative low density lipoprotein portion from a specimen. The present invention further proposes the use of a convenient biological kit to separate an electronegative low density lipoprotein portion from a specimen in the absence of using an organo-iodine additive.

The present invention in a first aspect proposes a glycerol-containing biological kit for the separation of electronegative low density lipoproteins from a specimen. The biological kit may include reagents, such as an optional first reagent, a second reagent, a third reagent and a fourth reagent in the absence of an organo-iodine additive, such as iodixanol. The optional first reagent may include water and may have a resistivity of at least 18 MΩ·cm. The second reagent may have a density from 1.090 g/cm³ to 1.093 g/cm³ and may include a first buffer, a second buffer and a salt. The third reagent may have a density from 1.060 g/cm³ to 1.063 g/cm³ and may include the first buffer, the second buffer, an organic compound and the salt. The fourth reagent may have a density from 0.98 g/cm³ to 1.001 g/cm³ and may include the first buffer and the second buffer.

In one embodiment of the present invention, the first buffer may include a Tris-base buffer which may have a concentration from 0.01M to 0.05M and a pH value from 7.0 to 9.0.

In another embodiment of the present invention, the second buffer may include an EDTA-base buffer which may have a concentration from 0.1 mM to 1 mM and a pH value from 7.0 to 9.0.

In another embodiment of the present invention, the organic compound may be selected from a group consisting of glycerol, a triol, sucrose and DMSO in a concentration from 0.5% (v/v, volume per volume) to 10% (v/v).

In another embodiment of the present invention, the salt may be selected from a group consisting of NaCl, KCl, KBr and CsI.

The present invention in a second aspect proposes a method for separating an electronegative low density lipoprotein from a specimen. The method may involve the use of the above-mentioned biological kit which includes several reagents without the need of an organo-iodine additive, such as iodixanol.

The method of the present invention may include the following operations. First, a specimen is provided. The specimen may include a mixture of lipoproteins. Second, a Reagent B, a Reagent C and a Reagent D are added to the specimen. The Reagent B may have a density from 1.090 g/cm³ to 1.093 g/cm³ and may include a first buffer, a second buffer and a salt. The Reagent C may have a density from 1.060 g/cm³ to 1.063 g/cm³ and may include the first buffer, the second buffer, an organic compound and the salt. The Reagent D may have a density from 0.98 g/cm³ to 1.001 g/cm³ and may include the first buffer and the second buffer. Then, the specimen may be subjected to an one-step first centrifugation at a first speed for at least 6 hours to obtain a sample. Following centrifugation, lipoprotein portions may be separated and removed according to their density in the absence of an organo-iodine additive, such as iodixanol, but the present invention is not limited thereto. For example, the first portion of the sample with a density from 0.960 g/cm³ to 1.006 g/cm³ may be removed from the sample. The second portion of the sample with a density from 1.006 g/cm³ to 1.019 g/cm³ may be removed from the sample. The third portion of the sample with a density from 1.019 g/cm³ to 1.031 g/cm³ may be removed from the sample. The fourth portion of the sample with a density from 1.031 g/cm³ to 1.036 g/cm³ may be removed from the sample. The fifth portion of the sample with a density from 1.036 g/cm³ to 1.063 g/cm³ may be removed from the sample. Next, at least one of the first portion, the second portion, the third portion, the fourth portion and the fifth portion may be subjected to a dialysis to obtain at least one purified lipoprotein portion corresponding to at least one of the first portion, the second portion, the third portion, the fourth portion and the fifth portion in the absence of an organo-iodine additive, such as iodixanol. At least one of the third portion, the fourth portion and the fifth portion may include an electronegative low density lipoprotein.

In one embodiment of the present invention, the method may further include the following operations. A second plasma may be obtained by subjecting a first plasma to a second centrifugation at a second plasma speed for 1 hour to 2 hours in the presence of a Reagent A. The Reagent A may include water and may have a resistivity of at least 18 MΩ·cm. The specimen may be obtained by removing the water and chylomicrons from the second plasma.

In another embodiment of the present invention, the method may further include the following operations. For example, a first plasma which is a biological supernatant of a biological substance may be obtained by subjecting the biological substance to a third centrifugation at a biological substance speed for 5 minutes to 15 minutes in the presence of a first agent to yield the biological supernatant.

In one embodiment of the present invention, the method may further include the following operations. At least one purified lipoprotein portion may be subjected to filtration before subjecting it to storage in the absence of an organo-iodine additive, such as iodixanol.

In another embodiment of the present invention, the method may further include the following operation. At least one purified lipoprotein portion may be subjected to electrophoresis for the analysis or verification of the at least one purified lipoprotein portion in the absence of an organo-iodine additive, such as iodixanol.

In another embodiment of the present invention, the first buffer may include a Tris-base buffer having a concentration from 0.01M to 0.05M and having a pH value from 7.0 to 9.0. Tris, a.k.a. tris(hydroxymethyl)aminomethane, molecular weight 121.14 g/mol.

In another embodiment of the present invention, the second buffer may include an EDTA-base buffer having a concentration from 0.1 mM to 1 mM and having a pH value from 7.0 to 9.0. EDTA, a.k.a. ethylenediaminetetraacetic acid, molecular weight 292.24 g/mol.

In another embodiment of the present invention, the salt may be a halide compound, such as selected from a group consisting of NaCl, KCl, KBr and CsI. A halide compound in the reagents of the present invention is selected from a group consisting of inorganic halide compounds, for example a fluoride, a chloride, a bromide and an iodide. An iodide compound in the reagents of the present invention is selected from a group consisting of inorganic iodide compounds, for example an iodide salt, such as CsI, but the present invention is not limited thereto.

In another embodiment of the present invention, the organic compound may be selected from a group consisting of glycerol, a triol, sucrose and DMSO. The organic compound may be a nitrogen-free organic compound or an iodine-free organic compound.

In another embodiment of the present invention, the organic compound may have a concentration from 0.5% (v/v) to 10% (v/v).

In another embodiment of the present invention, the method may further include the following operation. A sixth portion of the sample with a density from 1.063 g/cm³ to 1.210 g/cm³ may be removed from the sample. The sixth portion may include a high-density lipoprotein (HDL).

In another embodiment of the present invention, the second plasma speed may be in a range from 40000 rpm to 60000 rpm.

In another embodiment of the present invention, the biological substance speed may be in a range from 3000 rpm to 3600 rpm.

In another embodiment of the present invention, the first agent may be selected from a group consisting of EDTA, acid-citrate-dextrose and Heparin.

In another embodiment of the present invention, the relative centrifuge force may be in a range from 200 g to 300 g.

In another embodiment of the present invention, the time to subject the sample to the first centrifugation at the first speed may be not more than 24 hours.

In another embodiment of the present invention, the first speed may be in a range from 60000 rpm to 100000 rpm.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 shows an image of a sample in a tube which contains multiple portions corresponding to different lipoproteins from a given specimen within density distribution after the specimen centrifugation for 6 hours in accordance with the method of the present invention.

FIG. 2 shows an image of a sample in a tube which contains multiple portions corresponding to different lipoproteins from a given specimen within density distribution after the specimen centrifugation for 24 hours in accordance with the method of the present invention.

FIG. 3 shows an image of SDS-PAGE patterns of proteins in different portions which correspond to the results as shown in FIG. 1 in accordance with the sample after the dialysis treatment and the filtration treatment in accordance with the method of the present invention.

FIG. 4 shows an image of SDS-PAGE patterns of proteins in different portions which correspond to the results as shown in FIG. 2 in accordance with the sample after the dialysis treatment and the filtration treatment in accordance with the method of the present invention.

FIG. 5 shows an image of a sample in a tube which contains multiple portions corresponding to different lipoproteins from a given specimen within density distribution after the specimen centrifugation in accordance with the method of the present invention.

FIG. 6 shows an image of SDS-PAGE patterns of proteins in different portions in accordance with the method of the present invention.

### DETAILED DESCRIPTION

As one skilled in the art will understand, electronic equipment manufacturers may refer to a component by different names. This document does not intend to distinguish between components that differ in name but not function. In the following description and in the claims, the terms “include”, “comprise” and “have” are used in an open-ended fashion, and thus should be interpreted to mean “include, but not limited to”. When an element or layer is referred to as being “on” or “connected to” another element or layer, it may be directly on or directly connected to the other element or layer, or intervening elements or layers may be presented. Although terms such as first, second, third, etc., may be used to describe diverse constituent elements, such constituent elements are not limited by the terms. The terms are used only to discriminate a constituent element from other constituent elements in the specification. The claims may not use the same terms, but instead may use the terms first, second, third, etc. with respect to the order in which an element is claimed. Accordingly, in the following description, a first constituent element may be a second constituent element in a claim.

The present invention in a first aspect provides a biological kit. The biological kit may be used for the separation of electronegative low density lipoproteins from a specimen or a biological substance. The biological substance may be obtained from an animal, for example a human, a mouse, a hamster, a rabbit or a pig, but the present invention is not limited to this. The biological substance may be a body fluid of an animal, for example whole blood, cerebrospinal fluid (CSF), from a cell organelle or from a tissue, but the present invention is not limited to these.

The biological kit may include reagents, such as a first reagent, a second reagent, a third reagent and a fourth reagent. The optional first reagent may include water or consist essentially of water, for example purified water. The purified water may be 0.22 μm-filtered water, deionized water, distilled water, deionization and distilled water (DD water) or the combination thereof, but the present invention is not limited to these. The first reagent may have a larger resistivity of at least 18 MΩ·cm, for example 18 MΩ·cm or more. The volume of the first reagent is optional depending on the practice of the present invention.

The second reagent may have a density in a range from 1.090 g/cm³ to 1.093 g/cm³. The second reagent may include a first buffer, a second buffer and a salt. The first buffer, the second buffer and the salt may be optionally mixed to obtain a solution with a final concentration of 0.01M to 0.05M of the first buffer, 0.1 mM to 1 mM of the second buffer, and a density in a range from 1.090 g/cm³ to 1.093 g/cm³ for the practice of the present invention. The second reagent may have a pH value in a range from 7.0 to 9.0, preferably about 8.0. The volume of the second reagent is optional depending on the practice of the present invention.

The third reagent may have a density in a range from 1.060 g/cm³ to 1.063 g/cm³. The third reagent may include the first buffer, the second buffer, an organic compound and the salt. The first buffer, the second buffer, the organic compound and the salt may be optionally mixed to obtain a solution with a final concentration of 0.01M to 0.05M of the first buffer, 0.1 mM to 1 mM of the second buffer, and a density in a range from 1.060 g/cm³ to 1.063 g/cm³ for the practice of the present invention. In some embodiments, the organic compound may be selected from a group consisting of glycerol, a triol, sucrose and DMSO. The triol may be an organic compound which has 3 hydroxyl groups. In some embodiments, the organic compound may have a concentration from 0.5% (v/v) to 10% (v/v) based on the total reagent. The third reagent may have a pH value in a range from 7.0 to 9.0, preferably about 8.0.

The fourth reagent may have a density in a range from 0.98 g/cm³ to 1.001 g/cm³. The fourth reagent may include the first buffer and the second buffer. The first buffer and the second buffer may be optionally mixed to obtain the fourth reagent with a final concentration of 0.01M to 0.05M of the first buffer, 0.1 mM to 1 mM of the second buffer, and a density in a range from 0.98 g/cm³ to 1.001 g/cm³ for the practice of the present invention. The fourth reagent may have a pH value in a range from 7.0 to 9.0, preferably about 8.0.

The first buffer may be a basic buffer system, for example a nitrogen-containing buffer. In some embodiments, the first buffer may include a Tris-base buffer. The first buffer may have a concentration in a range from 0.01M to 0.05M, preferably about 0.02M, but the present invention is not limited to this. The first buffer may have a pH value in a range from 7.0 to 9.0, preferably about 8, but the present invention is not limited to this. The formulation of a Tris-base buffer is well known to a person having ordinary skill in the art so the details will not be elaborated here.

The second buffer may be a chelating buffer system, for example a carboxyl-containing buffer. In some embodiments, the second buffer may include an EDTA-base buffer. The second buffer may have a concentration in a range from 0.1 mM to 1 mM, preferably about 0.5 mM, but the present invention is not limited to this. The second buffer may have a pH value in a range from 7.0 to 9.0, preferably about 8, but the present invention is not limited to this. The formulation of an EDTA-base buffer is well known to a person having ordinary skill in the art so the details will not be elaborated here.

In some embodiments, the salt may include the combination of M⁺X⁻, for example the cation or the anion may be a monovalent ion. For example, the salt may include a halide salt, such as NaCl, KCl, KBr and CsI, preferably KBr, but the present invention is not limited to these.

In a biological kit, each reagent may be individually disposed in a container. For example, the optional first reagent in a first container, the second reagent in a second container, the third reagent in a third container and the fourth reagent in a fourth container. Further, the biological kit may optionally include a guide or a manual for the descriptions and the use of the biological kit.

The present invention in a second aspect provides a method for separating electronegative low density lipoproteins from a biological fluid. The method may involve the use of a biological kit for the separation of electronegative low density lipoproteins from a biological liquid without the need of using an organo-iodine additive, such as iodixanol. For example, the method may involve the use of the above-mentioned biological kit to separate electronegative low density lipoproteins from a biological liquid in a shorter period of time.

The method of the present invention may involve 3 procedures, for example, a pre-treatment procedure, a separation procedure and an analysis procedure, but the present invention is not limited to these. The biological fluid may be a crude liquid or a pre-treated liquid. If the biological fluid is a crude liquid, the method may start from the pre-treatment procedure, followed by the separation procedure. Alternatively, if the biological fluid is a pre-treated liquid, the method may start from the separation procedure, followed by the analysis procedure. The following descriptions start from the pre-treatment procedure in the presence of a crude liquid with no pre-treatment, but the present invention is not limited to these.

### Pre-Treatment Procedure

First, a biological substance is provided. The biological substance may be a crude biological fluid with no pre-treatment. The biological substance may include different lipoproteins in a form of a mixture, for example a very low-density lipoprotein (VLDL), an electronegative low density lipoprotein (LDL-), a low-density lipoprotein (LDL), an intermediate-density lipoprotein (IDL), or a high-density lipoprotein (HDL), but the present invention is not limited to these.

For example, the biological substance may be obtained from an animal, for example a human, a mouse, a hamster, a rabbit or a pig, but the present invention is not limited to this. The biological substance may be a body fluid of an animal, for example whole blood, cerebrospinal fluid (CSF), from a cell organelle or from a tissue, but the present invention is not limited to this. The biological substance may be optionally pre-treated, for example an incubated cell or a serum. A crude biological substance may further include a solid or blood cells so a pre-treatment is needed to yield a serum to exclude the solid or blood cells.

The biological substance may be treated to obtain the plasma. For example, the biological substance may be subjected to a first plasma centrifugation to obtain the plasma so the plasma may be supernatant of the biological substance. The first plasma centrifugation may be carried out at a biological substance speed for a time period in a range from 5 minutes to 15 minutes, but the present invention is not limited to this. The biological substance speed may be in a range from 3000 rpm to 3600 rpm, but the present invention is not limited to this.

The first plasma centrifugation may be carried out in the presence of a first agent to yield the biological supernatant. In other words, the first agent may be added to the biological substance to facilitate the operation of the first plasma centrifugation. The first agent may be an anticoagulant to prevent the coagulation of the biological substance. For example, the first agent may be selected from a group consisting of EDTA, acid-citrate-dextrose and Heparin.

Next, the plasma may be further treated to obtain a second plasma. For example, the plasma may be subjected to a second plasma centrifugation to obtain the second plasma, which is a chylomicron-free portion of the plasma. For example, the second plasma centrifugation may separate the plasma into two portions, such as a top portion and a bottom portion. The top portion may be transparent and/or milky-white due to the concentration of excessive water and/or chylomicrons. The bottom portion may show a golden yellow color by a visual identification because the bottom portion may be a lipoprotein-rich and chylomicron-poor portion due to the concentration of various lipoproteins coming from the biological substance. Accordingly, a specimen may be obtained by removing the water and the chylomicrons from the second plasma. For example, the specimen may be the bottom portion.

The second plasma centrifugation may be carried out at a set speed for a time period of 1 hour to 2 hours in the presence of a Reagent A, but the present invention is not limited to this. The set speed may be in a range from 40000 rpm to 60000 rpm. The Reagent A may include water or consist essentially of water, for example purified water. The purified water may be 0.22 μm-filtered water, deionized water, distilled water, deionization and distilled water (DD water) or the combination thereof, but the present invention is not limited to these. The Reagent A may have a resistivity of at least 18 MΩ·cm, for example 18 MΩ·cm or more. The volume of the Reagent A is subject to change depending on the practice of the present invention.

After the pre-treatment procedure, various lipoproteins in the biological substance may be concentrated and separated to form a lipoprotein-rich and chylomicron-poor portion, for example, the bottom portion of a golden yellow appearance after the treatment of the third plasma centrifugation. The golden yellow bottom portion may include different lipoproteins in a form of a mixture, for example a very low-density lipoprotein, an electronegative low density lipoprotein, a low-density lipoprotein, an intermediate-density lipoprotein, or a high-density lipoprotein, but the present invention is not limited to these, to serve as the specimen for use in the subsequent separation procedure.

### Separation Procedure

A mixture of different lipoproteins, for example a very low-density lipoprotein, an electronegative low density lipoprotein, a low-density lipoprotein, an intermediate-density lipoprotein, or a high-density lipoprotein, may serve as a specimen for use in the separation procedure. The specimen may be a lipoprotein-rich and chylomicron-poor plasma coming from a crude biological substance after a pre-treatment to facilitate the operation of the separation procedure.

First, a specimen is provided. The specimen may include an aqueous mixture of lipoproteins. For example, the specimen may include at least one of a very low-density lipoprotein, an electronegative low density lipoprotein, a low-density lipoprotein, an intermediate-density lipoprotein or a high-density lipoprotein, to have a golden yellow color.

Second, a Reagent B, a Reagent C and a Reagent D may be added to the specimen to facilitate the subsequent separation, preferably the reagents are sequentially added in order. For example, the Reagent B is firstly added, followed by the addition of the Reagent C, then the addition of the Reagent D.

The volume of the Reagent B, of the Reagent C and of the Reagent D with respect to the volume of the specimen may depend on the volume of the specimen. For example, the volume of the specimen may be 2 mL to 3 mL, but the present invention is not limited to this. The volume of the Reagent B may be 2 ml to 4 mL, but the present invention is not limited to this. The volume of the Reagent C may be 2 mL to 4 mL, but the present invention is not limited to this. The volume of the Reagent D may be 0.2 mL to 1 mL, but the present invention is not limited to this. Preferably, the volume of the specimen, of the Reagent B, of the Reagent C, and of the Reagent D may be in a ratio of 6:6:6:1, but the present invention is not limited to this.

The Reagent B may have a relatively higher density. For example, the Reagent B may have a density in a range from 1.090 g/cm³ to 1.093 g/cm³. The Reagent B may include a first buffer, a second buffer and a salt. The salt may be added to adjust the density of the Reagent B to a preferable range.

The Reagent C may have a density in a range from 1.060 g/cm³ 1.063 g/cm³. The Reagent C may include the first buffer, the second buffer, the salt and an organic compound. The presence of the organic compound may facilitate the separation of the lipoproteins within a shorter period of time in the operation of the subsequent centrifugation. The salt may be added to adjust the density of the Reagent C to a preferable range.

The Reagent D may have a relatively lower density. For example, the Reagent D may have a density in a range from 0.98 g/cm³ to 1.001 g/cm³. The Reagent D may include the first buffer and the second buffer.

The first buffer may be a basic buffer system, for example a nitrogen-containing buffer. In some embodiments, the first buffer may include a Tris-base buffer. The first buffer may have a concentration in a range from 0.01M to 0.05M, preferably about 0.02M, but the present invention is not limited to this. The first buffer may have a pH value in a range from 7.0 to 9.0, preferably about 8, but the present invention is not limited to this.

The second buffer may be a chelating buffer system, for example a carboxyl-containing buffer. In some embodiments, the second buffer may include an EDTA-base buffer. The second buffer may have a concentration in a range from 0.1 mM to 1 mM, preferably about 0.5 mM, but the present invention is not limited to this. The second buffer may have a pH value in a range from 7.0 to 9.0, preferably about 8, but the present invention is not limited to this.

The salt may include the combination of M⁺X⁻, for example the cation or the anion may be a mono-valent ion. For example, the salt may include NaCl, KCl, KBr and CsI, preferably KBr, for the construction of a density gradient for fine-tuning the optimal separation results, but the present invention is not limited to this.

The organic compound may be: 1) soluble in water, such that it may be miscible with water or completely miscible with water; 2) a liquid, for example a viscous, hygroscopic liquid; 3) a nitrogen-free organic compound or an iodine-free organic compound, to facilitate the subsequent centrifugation and result in a premium density gradient within a shorter period of operation time. The resultant premium density gradient may facilitate the separation of different lipoproteins in the mixture to form portions of better quality in accordance with density distribution along a given direction. The organic compound may be selected from a group consisting of glycerol, a triol, sucrose and DMSO, preferably glycerol, i.e. one of triols. The triol may be an organic compound which has 3 hydroxyl groups. The organic compound may have a concentration in a range from 0.5% (v/v) to 10% (v/v).

In a reagent solution having a buffer and a salt for separating a lipoprotein from a specimen by centrifugation, the present invention may propose the improvement to include an organic compound which is miscible with water and in the reagent solution, for example to result in a premium density gradient within a shorter period of operation time. The organic compound may be completely miscible with water, a liquid, for example a viscous, hygroscopic liquid, or a nitrogen-free organic compound or an iodine-free organic compound.

The combination of the first buffer, of the second buffer, of the salt and of the organic compound may adjust, or preferably optimize, the centrifugal condition for the separation of the lipoproteins in the mixture. For example, the layer combination of the first buffer, of the second buffer, of the salt and of the organic compound may form a better density distribution, or a premium density gradient, or preferably an optimal density gradient, to facilitate the separation with the help of the centrifugation to result in a shorter operation time.

Second, the specimen may be subjected to a specimen centrifugation in the presence of the Reagent B, the Reagent C and the Reagent D. The presence of the Reagent B, the Reagent C and the Reagent D in the specimen centrifugation may facilitate the formation of the premium density gradient within a much shorter period of operation time than what is needed in prior art. The operation time, for example at least 6 hours, is preferably in a range from 6 hours to 24 hours. The specimen centrifugation may be carried out at a third plasma centrifugation speed, for example from 60000 rpm to 100000 rpm. After the separation of the specimen centrifugation, the specimen may be processed to obtain a sample. The sample may contain multiple portions which correspond to different lipoproteins in the mixture in accordance with the density distribution along the resultant premium density gradient.

Each one of the multiple portions may have different density ranges. Different lipoproteins may be found in different portions. For example, a first portion of the sample may have a density in a range from 0.960 g/cm³ to 1.006 g/cm³ as a result of the specimen centrifugation after a suitable period of operation time, but the present invention is not limited to this. The first portion may correspond to a very low-density lipoprotein. The first portion may be colorless or pale yellow. The first portion is triglyceride-rich, and may also contain apolipoprotein B100, apolipoprotein CI, apolipoprotein CII, apolipoprotein CIII, apolipoprotein E, and electronegative VLDL. Notably, it has been shown that electronegative VLDL level is significantly increased in certain disease states, such as in metabolic syndrome.

A second portion of the sample may have a density in a range from 1.006 g/cm³ to 1.019 g/cm³ as a result of the specimen centrifugation after a suitable period of operation time, but the present invention is not limited to this. The second portion may correspond to an intermediate-density lipoprotein. The second portion may be colorless. The second portion may contain a triglyceride, a cholesterol, apolipoprotein B100 and apolipoprotein E.

A third portion of the sample may have a density in a range from 1.019 g/cm³ to 1.031 g/cm³ as a result of the specimen centrifugation after a suitable period of operation time, but the present invention is not limited to this. The third portion may correspond to a low-density lipoprotein. The third portion may be may be colorless or pale yellow. The third portion is cholesterol-rich, and may contain apolipoprotein A, apolipoprotein B-100, and electronegative LDL. It has been shown that electronegative LDL may contain higher levels of apolipoprotein CIII, apolipoprotein AI, apolipoprotein E, apolipoprotein J, paraoxonase 1, and platelet-activating factor acetylhydrolase than normal LDL. Notably, it has been shown that electronegative LDL level is significantly increased in certain disease states, such as Type II diabetes, metabolic syndrome and during acute ischemic events.

A fourth portion of the sample may have a density in a range from 1.031 g/cm³ to 1.036 g/cm³ as a result of the specimen centrifugation after a suitable period of operation time, but the present invention is not limited to this. The fourth portion may correspond to a low-density lipoprotein. The fourth portion may be golden yellow, for example. The fourth portion is cholesterol-rich, and may contain apolipoprotein A, apolipoprotein B-100, and an electronegative LDL. It has been shown that an electronegative LDL may contain higher levels of apolipoprotein CIII, apolipoprotein AI, apolipoprotein E, apolipoprotein J, paraoxonase 1, and platelet-activating factor acetylhydrolase than normal LDL. Notably, it has been shown that an electronegative LDL level is significantly increased in certain disease states, such as Type II diabetes, metabolic syndrome and during acute ischemic events.

A fifth portion of the sample may have a density in a range from 1.036 g/cm³ to 1.063 g/cm³ as a result of the specimen centrifugation after a suitable period of operation time, but the present invention is not limited to this. The fifth portion may correspond to a low-density lipoprotein. The fifth portion may be light yellow, for example. The fifth portion is cholesterol-rich but apolipoprotein A-poor, and may contain apolipoprotein B-100, and an electronegative LDL. It has been shown that an electronegative LDL may contain higher levels of apolipoprotein CIII, apolipoprotein AI, apolipoprotein E, apolipoprotein J, paraoxonase 1, and platelet-activating factor acetylhydrolase than normal LDL. Notably, it has been shown that an electronegative LDL level is significantly increased in certain disease states, such as Type II diabetes, metabolic syndrome and during acute ischemic events.

A sixth portion of the sample may have a density in a range from 1.063 g/cm³ to 1.210 g/cm³ as a result of the specimen centrifugation after a suitable period of operation time, but the present invention is not limited to this. The sixth portion may correspond to a high-density lipoprotein. The sixth portion may look reddish brown, for example. The sixth portion is cholesterol-rich, phospholipid-rich, and may contain apolipoprotein AI, apolipoprotein AII, apolipoprotein AIV, apolipoprotein CI, apolipoprotein CII, apolipoprotein E, and an electronegative HDL. Specifically, an electronegative HDL is rich in apolipoprotein CIII. Notably, it has been shown that electronegative HDL level is increased in certain disease states, such as Alzheimer's Disease.

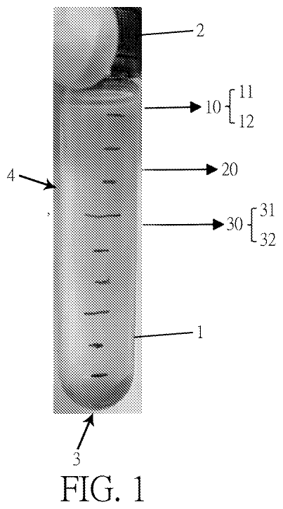

FIG. 1 shows an image of a sample in a tube which contains multiple portions corresponding to different lipoproteins from a given specimen within density distribution along the resultant premium density gradient after the specimen centrifugation for 6 hours in accordance with the method of the present invention. The total volume of sample 4 in the tube 1 is about 9.5 ml and contains 3 major layers, from the lid 2 to the bottom 3, namely a layer 10, a layer 20 and a layer 30 for example.

The layer 10 may have a density in a range from 1.000 g/cm³ to 1.0125 g/cm³ as a result of the specimen centrifugation. The layer 10 may include 2 portions. The top portion 11 of the layer 10 may have a volume about 200 μL. The bottom portion 12 of the layer 10 may be the balance of the layer 10 and may have a density about 1.0125 g/cm³. The layer 20 may be a colorless portion and may have a density about 1.025 g/cm³ as a result of the specimen centrifugation. The layer 30 may have a density in a range from 1.05 g/cm³ to 1.0625 g/cm³ as a result of the specimen centrifugation. The layer 30 may include 2 portions. The top portion 31 of the layer 30 may have a volume about 1 mL and have a golden yellow color. The bottom portion 32 of the layer 30 may be the balance of the layer 30, may have a density about 1.0625 g/cm³ and may have a yellow color.

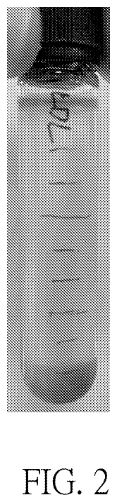

FIG. 2 shows an image of a sample in a tube which contains multiple portions corresponding to different lipoproteins from a given specimen within density distribution along the resultant premium density gradient after the specimen centrifugation for 24 hours in accordance with the method of the present invention. FIG. 2 shows an even clearer separation result.

After the specimen centrifugation for a suitable period of operation time, the following portions may be separately removed, preferably in the following order. For example, a first portion of the sample which may have a density in a range from 0.960 g/cm³ to 1.006 g/cm³ may be removed from the sample; a second portion of the sample which may have a density in a range from 1.006 g/cm³ to 1.019 g/cm³ may be removed from the sample; a third portion of the sample which may have a density in a range from 1.019 g/cm³ to 1.031 g/cm³ may be removed from the sample; a fourth portion of the sample which may have a density in a range from 1.031 g/cm³ to 1.036 g/cm³ may be removed from the sample; a fifth portion of the sample which may have a density in a range from 1.036 g/cm³ to 1.063 g/cm³ may be removed from the sample; a sixth portion of the sample which may have a density in a range from 1.063 g/cm³ to 1.210 g/cm³ may be removed from the sample, but the present invention is not limited to these.

A dialysis treatment may be carried out following the isolation of the target portions. Next, one or more of the first portion, the second portion, the third portion, the fourth portion, the fifth portion and the sixth portion may be subjected to a dialysis treatment. The dialysis treatment may remove excess salt from the portion to concentrate or to purify the lipoproteins in the portion without changing the substantial composition of the lipoproteins in the portion. The dialysis treatment may obtain a purified lipoprotein mixture corresponding to the first portion, the second portion, the third portion, the fourth portion, the fifth portion or the sixth portion with minimal salt content.

The dialysis treatment may reduce the salt content of the portions to a minimal. For example, a dialysis device may be used in the presence of the Reagent D to reduce the salt content of a portion and to reduce the electrical conductivity of a portion, for example the electrical conductivity of a portion is reduced to be less than 10 ms/au. A volume ratio of the portion to the reagent may be in a range from 1:100 to 1:500, preferably 1:500. The dialysis treatment may be carried out for 30 minutes to 60 minutes.

After the dialysis treatment, a filtration treatment may be further carried out. In some embodiments, one or more portions which include at least one purified lipoprotein mixture may be subjected to filtration before at least one purified lipoprotein portion is subjected to storage. The filtration treatment may remove bacteria from the purified lipoprotein portion to prevent contamination of the portion.

A hydrophilic syringe filter may be used to carry out the filtration treatment. For example, a hydrophilic syringe filter with a filter film, such as a 0.22 μm filter film of a suitable material may be used, but the present invention is not limited to this. A suitable material may include PVDF, Nylon, PES, MCE, CA, PTFE, GF, and PPhe for the filtration treatment, but the present invention is not limited to these.

After the filtration treatment, a filtered portion may be subjected to storage for a later use. A filtered portion may be kept at a lower temperature condition to facilitate the stable quality of the portion for storage. A lower temperature condition may be in a range from 4° C. to 10° C., preferably about 4° C.

In particular, lipoproteins which have a density in a range from 1.019 g/cm³ to 1.063 g/cm³ may include electronegative low density lipoproteins. For example, one or more of the third portion, of the fourth portion and of the fifth portion may include an electronegative low density lipoprotein mixture in a suitable density gradient. Alternatively, low density lipoproteins without apoprotein/apolipoprotein may correspond to the third portion of a density in a range from 1.019 g/cm³ to 1.030 g/cm³.

### Analysis Procedure

To verify the quality of, to study, or to investigate the lipoproteins after the separation procedure or to predict a person's condition, an analysis procedure may be carried out subsequent to the separation procedure. It is suggested that an apolipoprotein E positive result or an apolipoprotein CIII positive result correlates with a higher electronegative LDL level. For example, one or more purified lipoprotein mixture may be subjected to electrophoresis for the analysis or the verification of the quality of one or more portions. The electrophoresis for use in the verification of the quality may be a conventional gel electrophoresis. A conventional gel electrophoresis may be a polyacrylamide gel electrophoresis, for example by a 4% to 12% SDS-PAGE, preferably by a 10% SDS-PAGE. SDS-PAGE is an electrophoresis method which allows proteins to be separated by mass, preferably with the help of a marker. Because SDS-PAGE is well known to a person having ordinary skill in the art, the details are not elaborated here.

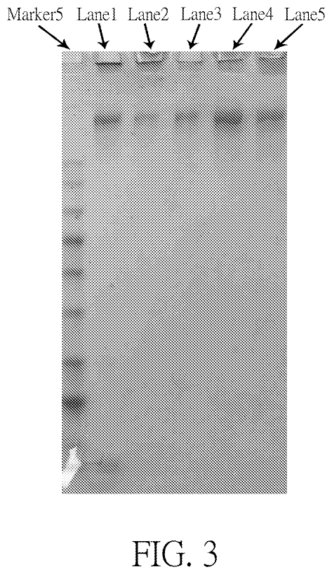

FIG. 3 shows an image of SDS-PAGE patterns of proteins in different portions which correspond to the results as shown in FIG. 1 in accordance with the sample after the dialysis treatment and the filtration treatment with the centrifugation operation for 6 hours. Lane 1 refers to the top portion 11, Lane 2 refers to the bottom portion 12, Lane 3 refers to the layer 20, Lane 4 refers to the top portion 31, and Lane 5 refers to the bottom portion 32 along with the apolipoprotein marker 5. For example, Lane 1 may correspond to VLDL, Lane 2 may correspond to IDL, Lane 3 may correspond to LDL-1, Lane 4 may correspond to LDL-2, and Lane 5 may correspond to LDL-3.

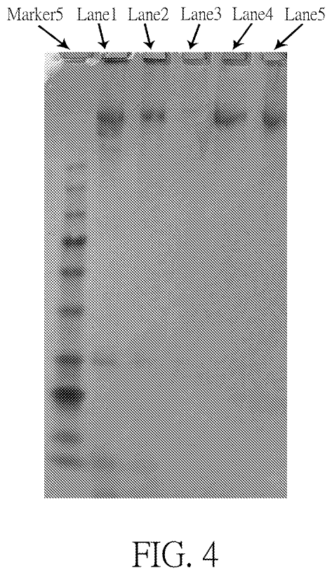

FIG. 4 shows an image of SDS-PAGE patterns of proteins in different portions which correspond to the results as shown in FIG. 2 in accordance with the sample after the dialysis treatment and the filtration treatment with the centrifugation operation for 24 hours. Lane 1 refers to the top portion 11, Lane 2 refers to the bottom portion 12, Lane 3 refers to the layer 20, Lane 4 refers to the top portion 31, and Lane 5 refers to the bottom portion 32 along with the apolipoprotein marker 5. For example, Lane 1 may correspond to VLDL, Lane 2 may correspond to IDL, Lane 3 may correspond to LDL-1, Lane 4 may correspond to LDL-2, and Lane 5 may correspond to LDL-3. FIG. 4 shows a better separation result between the top portion 11 and the bottom portion 12.

### Example

1. Whole blood of 5 mL in a blood collection tube with anticoagulant EDTA was subjected to a first plasma centrifugation (3000 rpm, 4° C. for 10 minutes) to obtain the first supernatant (2 mL, plasma of the whole blood).
2. 7 mL of the Reagent A was slowly added to the plasma from Step 1 in an ultra-speed centrifuge tube (polycarbonate material with cap, 16×76 mm, capacity 10.4 mL), then the tube was subjected to a second plasma centrifugation (45000 rpm for 2 hours with 90Ti rotor and Beckman L-100k) to remove chylomicron and water to obtain the chylomicron-poor plasma (3 mL, golden yellow).
3. 3 mL of Reagent B (0.02M Tris-base buffer, 0.5 mM EDTA buffer, KBr at pH 8.0, d=1.090 g/cm³), 3 mL of Reagent C (0.02M Tris-base buffer, 0.5 mM EDTA buffer, 1% glycerol (v/v), KBr at pH 8.0, d=1.063 g/cm³), 3 mL of the chylomicron-poor plasma, and 0.5 ml of Reagent D (0.02M Tris-base buffer, 0.5 mM EDTA buffer at pH 8.0, d=1.001 g/cm³), were sequentially added to a new ultra-speed centrifuge tube (polycarbonate material with cap, 16×76 mm, capacity 10.4 mL). Next, the tube was subjected to a third plasma centrifugation (65000 rpm, 4° C. respectively for 6 hours or 24 hours with 90Ti rotor and Beckman L-100k) to obtain the specimen (2.5 mL, pale yellow, light yellow to golden yellow). The result images are shown in FIG. 1 (6 hours) and in FIG. 2 (24 hours).
4. Portions with a density in a range from 1.019 g/cm³ to 1.063 g/cm³ in the specimen were separately removed for the dialysis treatment. These portions were injected to a dialysis device in the presence of the Reagent A (1×, sample:reagent=1:500 for 30 minutes till the electrical conductivity less than 10 ms/au) to obtain desalted portions (2.5 mL, pale yellow, light yellow to golden yellow).
5. A sterile syringe of 3 mL was used to remove the desalted portions carefully and connected to a filter (0.22 μm PVDF hydrophilic syringe filter) for the filtration. The desalted portions were collected in a glass vial (glass vial with none-cut lectra-bond cap and preslit PTEF/silicon septa, 12×32 mm, 8 mm screw neck, capacity 1.5 mL) to obtain a desalted, filtered and purified lipoprotein portion which includes an electronegative low density lipoprotein portion.
6. An optional 10% SDS-PAGE was used to verify the quality of the above procedures, or to predict a person's condition in the presence of the apolipoprotein marker. It is suggested that an apolipoprotein E positive result or an apolipoprotein CIII positive result correlates with a higher L5 LDL level. The result images are shown in FIG. 3 (6 hours) and in FIG. 4 (24 hours).

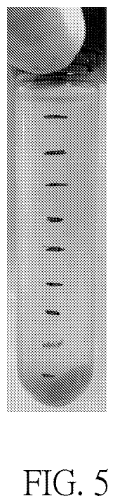

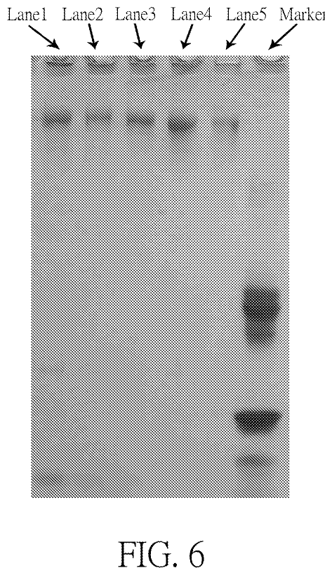

FIG. 5 shows an image of a sample in a tube which contains multiple portions corresponding to different lipoproteins from a given specimen within density distribution along the resultant premium density gradient after the specimen centrifugation in accordance with the method of the present invention. FIG. 6 shows an image of SDS-PAGE patterns of proteins in different portions. The treatment conditions of the two figures are identical.

The SDS-PAGE results may more rapidly imply a positive result or a negative result. As shown in FIG. 6, Lane 1 of sample 1 comes from a patient with a higher electronegative LDL level (3.1%). Lane 2 to Lane 5 come from patients with a lower electronegative LDL level (<1.6%). It has been previously established that the reference range for electronegative LDL level in healthy adults may be less than 1.6%. Evidently, the band in Lane 1 indicates an abnormally high electronegative LDL level. Higher electronegative LDL level has been linked with many diseases, such as atherosclerotic cardiovascular disease. To precisely quantify electronegative LDL levels, further isolation may be required to determine the details of the electronegative LDL mixtures. Lane 6 corresponds to apolipoprotein markers. The LDL samples coming from a rapid separation method are further subjected to the SDS-PAGE separation to isolate apolipoproteins of different molecular weights. It is suggested that an apolipoprotein E positive result or an apolipoprotein CIII positive result may possibly correlate with a higher electronegative LDL level.

The first plasma centrifugation may be carried out for 10 minutes. The second plasma centrifugation may be carried out for 1 hour. The specimen centrifugation may be carried out for 6 hours. The dialysis treatment may be carried out for 30 minutes. In other words, the present invention provides a quick method to separate a purified lipoprotein portion from a biological substance, such as whole blood, within a greatly reduced operation time of 7 hour 40 minutes. Further, the present invention provides a quick method to separate a purified lipoprotein portion, such as a chylomicron-free and cell-free serum, from a specimen within a reduced operation time of 6 hours. Accordingly, the present invention is advantageous in providing quicker methods to purify or to separate a purified lipoprotein portion including an electronegative low density lipoprotein portion from a source, such as whole blood or a serum, to greatly reduce the operation time.

Any arbitrary combination of the parameters of the embodiments may be optionally selected to facilitate the practice of the present invention.

The numeral value ranges within the maximum and minimum values obtained from the combination ratio relationships of the parameters disclosed in the specification of the invention may all be implemented accordingly.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A glycerol-containing biological kit for the separation of electronegative low density lipoproteins from a specimen, comprising:
a first reagent having a density from 1.090 g/cm³ to 1.093 g/cm³ and comprising a first buffer, a second buffer and a salt;
a second reagent having a density from 1.060 g/cm³ to 1.063 g/cm³ and comprising the first buffer, the second buffer, glycerol and the salt; and
a third reagent having a density from 0.98 g/cm³ to 1.001 g/cm³ and comprising the first buffer and the second buffer.

2. The glycerol-containing biological kit of claim 1, wherein the second buffer is a Tris-base buffer which has a concentration from 0.01M to 0.05M and a pH value from 7.0 to 9.0.

3. The glycerol-containing biological kit of claim 1, wherein the third buffer is an EDTA-base buffer which has a concentration from 0.1 mM to 1 mM and a pH value from 7.0 to 9.0.

4. The glycerol-containing biological kit of claim 1, wherein the glycerol has a concentration from 0.5% (v/v, volume per volume) to 10% (v/v) based on the third reagent.

5. The glycerol-containing biological kit of claim 1, wherein the salt is selected from a group consisting of NaCl, KCl, KBr and CsI.

6. The glycerol-containing biological kit of claim 1, further comprising:
a fourth reagent comprising water and having a resistivity of at least 18 MΩ·cm.

7. A method for separating an electronegative low density lipoprotein from a specimen, comprising:
providing the specimen which comprises a mixture of lipoproteins;
adding a Reagent B, a Reagent C and a Reagent D to the specimen before subjecting the specimen to a first centrifugation at a first speed for at least 6 hours to obtain a sample, wherein the Reagent B has a density from 1.090 g/cm³ to 1.093 g/cm³ and comprises a first buffer, a second buffer and a salt, the Reagent C has a density from 1.060 g/cm³ to 1.063 g/cm³ and comprises the first buffer, the second buffer, glycerol and the salt, and the Reagent D has a density from 0.98 g/cm³ to 1.001 g/cm³ and comprises the first buffer and the second buffer;
removing a first portion of the sample and with a density from 0.960 g/cm³ to 1.006 g/cm³ from the sample;
removing a second portion of the sample and with a density from 1.006 g/cm³ to 1.019 g/cm³ from the sample;
removing a third portion of the sample and with a density from 1.019 g/cm³ to 1.031 g/cm³ from the sample;
removing a fourth portion of the sample and with a density from 1.031 g/cm³ to 1.036 g/cm³ from the sample;
removing a fifth portion of the sample and with a density from 1.036 g/cm³ to 1.063 g/cm³ from the sample; and
subjecting at least one of the first portion, the second portion, the third portion, the fourth portion and the fifth portion to a dialysis to obtained at least one purified lipoprotein mixture corresponding to at least one of the first portion, the second portion, the third portion, the fourth portion and the fifth portion, wherein at least one of the third portion, the fourth portion and the fifth portion comprises an electronegative low density lipoprotein.

8. The method of claim 7, further comprising:
obtaining a second plasma by subjecting a first plasma to a second centrifugation at a second plasma speed for 1 hour to 2 hours in the presence of a Reagent A, wherein the Reagent A comprises water and having a resistivity of at least 18 MΩ·cm; and
obtaining the specimen by removing the water and chylomicrons from the second plasma.

9. The method of claim 8, further comprising:
obtaining a first plasma which is a biological supernatant of a biological substance by subjecting the biological substance to a third centrifugation at a biological substance speed for 5 minutes to 15 minutes in the presence of a first agent to yield the biological supernatant.

10. The method of claim 7, further comprising:
subjecting the at least one purified lipoprotein mixture to filtration before subjecting the at least one purified lipoprotein mixture to storage.

11. The method of claim 7, further comprising:
subjecting the at least one purified lipoprotein mixture to electrophoresis for the analysis of the at least one purified lipoprotein mixture.

12. The method of claim 7, wherein the first buffer is a Tris-base buffer having a concentration from 0.01M to 0.05M and having a pH value from 7.0 to 9.0.

13. The method of claim 7, wherein the second buffer is an EDTA-base buffer having a concentration from 0.1 mM to 1 mM and having a pH value from 7.0 to 9.0.

14. The method of claim 7, wherein the salt is selected from a group consisting of NaCl, KCl, KBr and CsI.

15. The method of claim 7, wherein glycerol is in a concentration from 0.5% (v/v) to 10% (v/v) based on the third reagent.

16. The method of claim 7, further comprising:
removing a sixth portion of the sample with a density from 1.063 g/cm³ to 1.210 g/cm³ from the sample, wherein the sixth portion comprises a high-density lipoprotein.

17. The method of claim 8, wherein the second plasma speed is in a range from 40000 rpm to 60000 rpm.

18. The method of claim 9, wherein the biological substance is whole blood, the biological substance speed is in a range from 3000 rpm to 3600 rpm, and the first agent is selected from a group consisting of EDTA, acid-citrate-dextrose and Heparin.

19. The method of claim 7, wherein subjecting the sample to the first centrifugation at the first speed for not more than 24 hours.

20. The method of claim 7, wherein the first speed is in a range from 60000 rpm to 100000 rpm.
